(19) United States
(12) Patent Application Publication
(10) Pub. No.: US 2009/0156473 A1
(43) Pub. Date: Jun. 18, 2009

(54) PHARMACEUTICAL COMPOSITION FOR THE TREATMENT OF VIRAL INFECTIONS AND/OR TUMOR DISEASES BY INHIBITING PROTEIN FOLDING AND PROTEIN BREAKDOWN

(75) Inventor: Ulrich Schubert, Jena (DE)

Correspondence Address: OBLON, SPIVAK, MCCLELLAND MAIER & NEUSTADT, P.C., 1940 DUKE STREET, ALEXANDRIA, VA, 22314, US

(73) Assignee: Virologik GmbH, Erlangen (DE)

(21) Appl. No.: 12/325,598

(22) Filed: Dec. 1, 2008

Related U.S. Application Data
(63) Continuation of application No. PCT/EP2007/005542, filed on Jun. 1, 2007.

(30) Foreign Application Priority Data
Jun. 1, 2006 (DE) 10 2006 026 464.9

(51) Int. Cl. A61K 38/13; A61K 31/436; A61K 38/00

(52) U.S. Cl. 514/11; 514/291; 514/2; 514/19

(57) ABSTRACT

The invention relates to the treatment of viral diseases with at least one proteasome inhibitor and one inhibitor of protein-folding enzymes.

### CROSS-REFERENCE TO RELATED APPLICATIONS

This application is a continuation of PCT/EP2007/055425 filed Jun. 1, 2007 and claims the benefit of DE 10 2006 026 464.9 filed Jun. 1, 2006, both of which are incorporated herein by reference.

### FIELD OF THE INVENTION

The invention relates to a pharmaceutical composition that contains at least one proteasome inhibitor and one inhibitor of protein-folding enzymes as active components. These agents are suitable for treatment of acute and chronic infections by viruses pathogenic for humans and animals. Such viruses include in particular pathogens of infectious diseases such as AIDS, hepatitis, hemorrhagic fever, SARS, smallpox, measles, polio or flu. Subject matter of the invention are agents that on the one hand contain inhibitors of protein folding as active ingredients. They include inhibitors of cellular folding enzymes (the enzyme chaperones) as well as substances that interfere with protein folding by chemical chaperones. On the other hand, these agents contain components that interfere with the ubiquitin-proteasome system, especially agents that inhibit the 26S proteasome. By combining these therapeutic agents, it may be possible to interfere with the efficiency of protein biosynthesis and the degradation of improperly folded proteins, separately of each other or simultaneously. In the sum of these effects, it may also be possible systematically to impair the viability of degenerated tumor cells and/or cells infected acutely and/or chronically by viruses and thus to direct them to programmed cell death (apoptosis). Areas of application are the treatment of viral infections and/or tumor diseases.

### DESCRIPTION OF THE RELATED ART

Inhibitors of protein-folding enzymes are known from WO 2005/063281 A2.

Proteasome inhibitors have been described both for treatment of tumor diseases (for example, U.S. Pat. No. 6,083,903) and also for treatment of viral infections (WO 02/30455).

Heretofore a combination of inhibitors of protein-folding enzymes and proteasome inhibitors has not been described. Only the combination of protease inhibitors that are not selective for proteasomes with inhibitors of protein-folding enzymes was mentioned in WO 2005/063281 A2.

### SUMMARY OF THE INVENTION

The object of the invention was to provide new pharmaceutical compositions for treatment of viral infections and/or tumor diseases.

### BRIEF DESCRIPTION OF THE DRAWINGS

FIG. 1: Up to a concentration of 10 nM, the Hsp90 inhibitor 17-AAG does not exhibit any cytotoxicity in CEM cells. CD4⁺ T lymph cells (CEM cells) were incubated with various concentrations of 17-AAG and the time-dependent color change, which corresponds to the number of viable cells, was determined by means of fluorescence measurement after addition of AlamarBlue™ (Invitrogen).

FIG. 2: Under the influence of 17-AAG, HeLaSS6 cells transfected with subgenomic HIV-1 expression vector pNLenv1 exhibit reduced Gag processing in the virus fraction and intensified Hsp70 expression in the cell fraction.

FIG. 3: Antiviral effect of 17-AAG alone and also in combination with the proteasome inhibitor PS341 versus X4-trophic HI viruses in the HLAC model, plotted on the basis of the RT data of the respective kinetic points of two different tonsils (A and B).

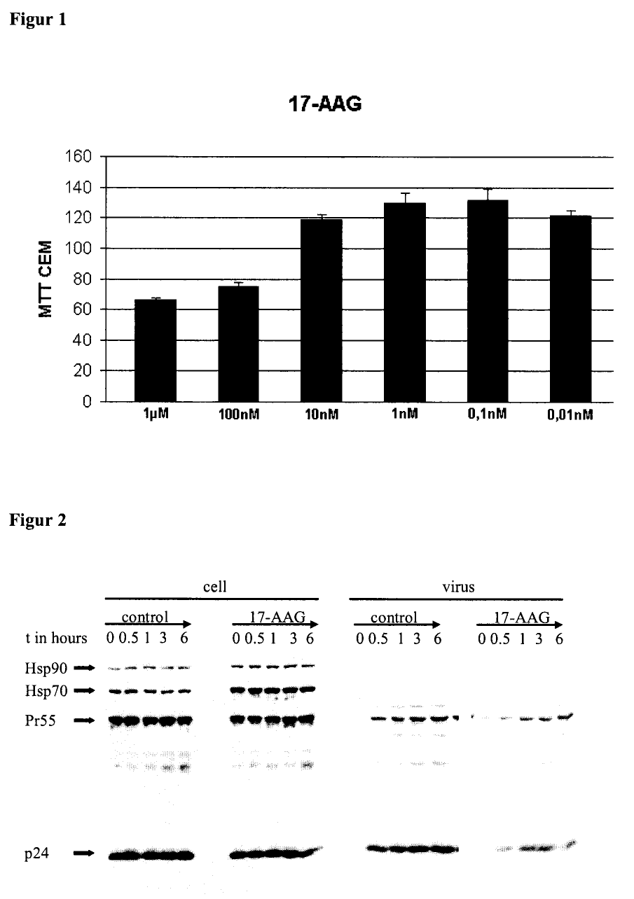

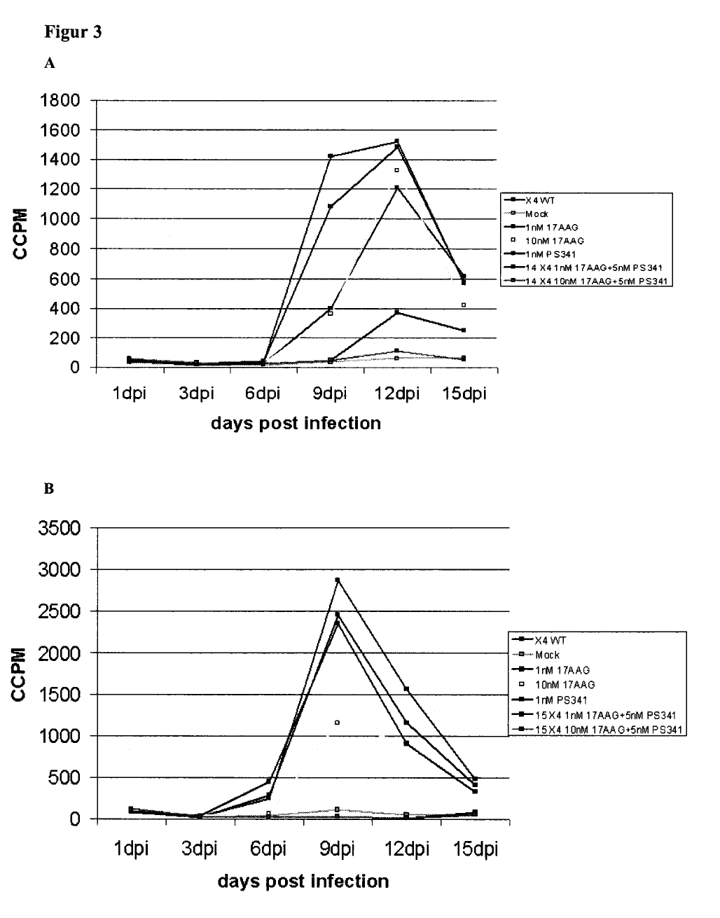

Virus replication of the X4-trophic HI viruses in tonsil A (A) was not clearly influenced either by incubation with 1 nM proteasome inhibitor PS341, 1 nM 17-AAG or 10 nM 17-AAG. Only the combination of the two substances (5 nM PS341 and 1 nM 17-AAG) achieved a clear decrease of virus replication. In this connection, it was found that this additive effect during application of both substances can be further potentiated by a higher concentration of the Hsp90 inhibitor 17-AAG (10 nM). Tonsil B (B) also did not exhibit any influence on X4-trophic HIV replication during incubation with 1 nM PS341 or 1 nM 17-AAG. In contrast to tonsil A, a distinct reduction of virus replication in tonsil B was already found by addition of 10 nM 17-AAG. For all combinations of proteasome inhibitor PS341 with Hsp90 inhibitor 17-AAG, it was no longer possible to detect any virus replication whatsoever.

### DETAILED DESCRIPTION OF THE INVENTION

The object was achieved according to the features of the claims. The inventive combination of inhibitors of protein-folding enzymes and proteasome inhibitors is superior to the prior art. According to the invention, there has been provided a pharmaceutical composition that contains at least one inhibitor of the ubiquitin-proteasome system and one inhibitor of protein-folding systems as active components, or a method for influencing protein folding.

The inhibitor of protein-folding enzymes is preferably at least one inhibitor of cellular chaperones or at least one chemical substance that directly influences protein folding (chemical anti-chaperone).

Local hyperthermia is preferably used as a method for influencing protein folding.

A further preferred embodiment of the invention comprises using, as inhibitors of cellular chaperones or of chemical anti-chaperones, substances that

a) inhibit, regulate or otherwise influence the folding and proteolytic maturation of virus proteins and thereby inhibit the release and replication of viruses, especially of pathogens of infectious diseases such as AIDS, hepatitis, hemorrhagic fever, SARS, smallpox, measles, polio, herpes viral infections or flu, or

b) interfere with the proliferation of degenerate cells, especially tumor cells, by directing them to programmed cell death due to accumulation of incorrectly folded proteins.

The inventive pharmaceutical composition is characterized in that there are used, as inhibitors of cellular chaperones or of chemical anti-chaperones, substances that especially influence the enzymatic activities of molecular folding enzymes of the host cells. The cells of higher eukaryotes absorb these inhibitors or substances and, after cell absorption, block the protein folding of viral structural proteins and of proteins from tumor cells. The inhibitors or substances can be administered in vivo in various oral, intravenous, intramuscular or subcutaneous forms, or in encapsulated form, with or without changes that carry cell specificity, have low cytotoxicity by virtue of the use of a well-defined application and/or dosage regimen, trigger no or only slight side effects, have a relatively long metabolic half life and exhibit a relatively slow clearance rate in the organism.

The inventive pharmaceutical composition is further characterized in that there are used, as inhibitors of cellular chaperones or of chemical anti-chaperones, substances that

a) are isolated in natural form from microorganisms or other natural sources, or

b) are formed from natural substances by chemical modifications, or

c) are produced by completely synthetic methods, or

d) are synthesized in vivo by gene therapeutic methods.

The inhibitors of cellular chaperones or the chemical anti-chaperones interfere with the highly organized processes of assembly and proteolytic maturation of viral structural proteins and thereby suppress the release and production of infectious progeny viruses. Moreover, these substances regulate, interfere with or block the folding of viral proteins and/or of tumor-specific proteins by interfering with the late processes of virus replication, such as assembly, budding, proteolytic maturation and virus release. The proteolytic processing of precursor proteins of viral polyproteins is thereby interfered with. Moreover, the activity of viral proteases is blocked.

A further preferred embodiment of the invention comprises using, as inhibitors of cellular chaperones or of chemical anti-chaperones, substances that interfere with the activities of cellular proteases and/or of enzymes, such as ligases, kinases, hydrolases, glycosylation enzymes, phosphatases, DNAses, RNAses, helicases and transferases, which are involved in virus maturation. The inventive inhibitors of cellular chaperones or the chemical anti-chaperones possess a broad range of action and can therefore be used as novel broad-spectrum virostatics for prevention and/or for therapy of different viral infections.

The pharmaceutical composition is characterized in that there are used, as inhibitors of cellular chaperones or of chemical anti-chaperones, substances that block or inhibit cellular chaperones such as heat shock proteins (hsp), especially the activities of the Hsp27, Hsp30, Hsp40, Hsp60, Hsp70, Hsp72, Hsp73, Hsp90, Hsp104 and Hsc70 heat shock proteins.

As inhibitors of cellular chaperones there can be used substances that belong to the following substance classes and their derivatives: geldanamycin (inhibits Hsp90), radicicol (tyrosine kinase inhibitor; inhibits Hsp90), deoxyspergualin (inhibits Hsc70 and Hsp90), 4-PBA (4-phenyl butyrate; downregulation of protein and mRNA expression of Hsc70), herbimycin A (tyrosine kinase inhibitor with Hsp72/73 induction), epolactaene (inhibitor of Hsp60), Scythe and Reaper (inhibit Hsp70), artemisinin (inhibitor of Hsp90), CCT0180159 (as a pyrazole inhibitor of Hsp90) and SNX-2112 (Hsp90 inhibitor), radanamycin (macrolid chimera of radicicol and geldanamycin), novobiocin (Hsp90 inhibitor), quercetin (inhibitor of Hsp70 expression).

As chemical anti-chaperones there can be used substances that regulate, interfere with or block the protein conformation and folding of viral and/or tumor-specific proteins. They include substances such as glycerol, trimethylamine, betaine, trehalose or deuterated water (D₂O). Furthermore, there can be used substances that are suitable for the treatment, therapy and inhibition of infections with different viruses that are pathogenic for humans or animals, or substances that are suitable for the treatment, therapy and inhibition of infections with pathogens of chronic infectious diseases such as AIDS (HIV-1 and HIV-2), of hepatitis (HCV and HBV), of the pathogen of “Severe Acute Respiratory Syndrome” (SARS), or in other words the SARS CoV (corona virus), of smallpox viruses, of pathogens of viral hemorrhagic fever (VHF), such as the Ebola viruses, which are representatives of the Filoviridae family, and of flu pathogens such as the influenza A virus. They include, for example, cyclosporin A and/or tacrolimus.

The inventive pharmaceutical composition is further characterized in that the UPS inhibitors comprise at least one substance that

a) in the form of proteasome inhibitors especially influences the enzymatic activities of the complete 26S proteasome complex and of the free 20S catalytically active proteasome structure that is not assembled with regulatory subunits, or

b) especially inhibits the action of ubiquitin ligases, or

c) especially inhibits the action of ubiquitin hydrolases, or

d) especially inhibits the action of ubiquitin-activating enzymes, or

e) especially inhibits the mono-ubiquitinylation of proteins, or

f) especially inhibits the poly-ubiquitinylation of proteins.

The proteasome inhibitors are absorbed by higher eukaryotes and, after cell absorption, interact with the catalytic subunits of the proteasome and thus block all or individual proteolytic activities of the proteasome—the trypsin, the chymotrypsin and/or the postglutamyl peptide hydrolyzing activities—within the 26S or even the 20S proteasome complex irreversibly or reversibly.

As proteasome inhibitors there are used substances that

a) are isolated in natural form from microorganisms or other natural sources, or

b) are formed from natural substances by chemical modifications, or

c) are produced by completely synthetic methods, or

d) are synthesized in vivo by genetic therapy methods, or

e) are produced in vitro by genetic engineering methods, or

f) are produced in microorganisms.

The proteasome inhibitors are compounds that belong to the following substance classes:

a) naturally occurring proteasome inhibitors:
peptide derivatives that contain C-terminal epoxyketone structures, or
β-lactone derivatives, or
aclacinomycin A (also known as aclarubicin), or
lactacystine and its chemical modified variants, such as the cell membrane-penetrating variant “clasto-lactacysteine β-lactone”

b) synthetically produced proteasome inhibitors:
modified peptide aldehydes such as N-carbobenzoxy-L-leucinyl-L-leucinyl-L-leucinal (also known as MG132 or zLLL), its boric acid derivative MG232; N-carbobenzoxy-Leu-Leu-Nva-H (designated MG115; N-acetyl-L-leucinyl-L-leucinyl-L-norleucinal (designated LLnL), N-carbobenzoxy-Ile-Glu(OBut)-Ala-Leu-H (also known as PSI);

c) peptides that contain a C-terminal α,β-epoxyketone structures, and also vinylsulfones such as carbobenzoxy-L-leucinyl-L-leucinyl-L-leucine vinylsulfone or 4-hydroxy-5-iodo-3-nitrophenylactetyl-L-leucinyl-L-leucinyl-L-leucine vinylsulfone (NLVS);

d) glyoxalic or boric acid groups such as
pyrazyl-CONH(CHPhe)CONH(CHisobutyl)B(OH)₂) as well as
dipeptidyl-boric acid derivatives or

e) pinacol esters such as benzyloxycarbonyl(Cbz)-Leu-Leu-boroLeu pinacol esters.

Particularly suitable proteasome inhibitors are the epoxyketones epoxomicin (epoxomycin, molecular formula: C₂₈H₈₆N₄O₇) and/or eponemycin (eponemicin, molecular formula: C₂₀H₃₆N₂O₅) or proteasome inhibitors from the PS series the compounds:

a) PS-519 as the β-lactone and also as the lactacystine derivative the compound 1R-[1S,4R,5S]]-1-(1-hydroxy-2-methylpropyl)-4-propyl-6-oxa-2-azabicyclo[3.2.0]heptane-3,7-dione, molecular formula C₁₂H₁₉NO₄, and/or

b) PS-314 as the peptidyl boric acid derivative the compound N-pyrazinecarbonyl-L-phenylalanin-L-leucine boric acid, molecular formula C₁₉H₂₅BN₄O₄, and/or

c) PS-273 (morpholin-CONH—(CH-naphthyl)-CONH—(CH-isobutyl)-B(OH)₂) and its enantiomer PS-293, and/or

d) the compound PS-296 (8-quinolyl-sulfonyl-CONH—(CH-napthyl)-CONH(—CH-isobutyl)-B(OH)₂), and/or

e) PS-303 (NH₂(CH-naphthyl)-CONH—(CH-isobutyl)-B(OH)₂), and/or

f) PS-321 as (morpholin-CONH—(CH-napthyl)-CONH—(CH-phenylalanin)-B(OH)₂), and/or

g) PS-334 (CH₃—NH—(CH-naphthyl-CONH—(CH-isobutyl)-B(OH)₂), and/or

h) the compound PS-325 (2-quinol-CONH—(CH-homo-phenylalanin)-CONH—(CH-isobutyl)-B(OH)₂), and/or

i) PS-352 (phenyalanin-CH₂—CH₂—CONH—(CH-phenylalanin)-CONH—(CH-isobutyl)-1-B(OH)₂), and/or

j) PS-383 (pyridyl-CONH—(CHρF-phenylalanin)-CONH—(CH-isobutyl)-B(OH)₂)

are used.

The described pharmaceutical compositions are suitable as medicinal products or for production of agents for treatment of viral infections and/or tumor diseases. Combination with other agents for treatment of viral infections and/or tumor diseases is also possible.

These agents may be used according to the invention in the form of
inhalations
depot forms
plasters
in microelectronic systems (“intelligent pills”)

Also possible is use in oncology and/or oncology and virology for treatment of
glioblastoma (malignant brain tumors)
breast CA (CA=cancer)
head, neck CA
squamous—platelet epithelial CA
ovarian CA
bronchial CA (small-cell, large-cell)
thyroid CA
lung CA
colon CA
pancreatic CA
leukemia (AML, ALL, CML, CLL)
acute myeloic, chronic

acute lymphatic, chronic
lymphoma (non-Hodgkins)
cervical Ca
neuroblastoma
skin CA (melanoma)
prostate CA
bladder CA
sarcoma (bone and pulp)
diaphragm CA
gastrointestinal CA (such as stomach, esophagus)
testicular Ca
metastases (such as bone marrow)
lymphoma viruses
herpes simplex
cytomegaly
chicken pox
varicella zoster
measles
Lassa fever
AIDS
mumps (-meningitis, -orchitis)
enteritis; flu (all forms)
encephalitis
hepatitis (A, B, C, D, E, G)
German measles
Coxsackie B
polio (-myelitis)
encephalomyelitis
pancreatitis
pneumonia
myocarditis
tropical diseases (viral)
all double-strand and single-strand DNA and RNA viruses that are pathogenic for humans.

Surprisingly, it has been found that proteins with extensive deficient folding are formed by interference with the protein-folding mechanisms. These deficient products of protein biosynthesis are normally degraded by the ubiquitin-proteasome system (UPS) and thus are removed from the cell metabolism. During inhibition of the UPS, for example by proteasome inhibitors and/or by inhibitors of ubiquitin ligases, these deficient products of protein biosynthesis, which are usually poly-ubiquitinylized and improperly folded, accumulate in the cell and thereby trigger diverse interferences with the cell metabolism. The sum of the effects of these interferences will direct the cell in question preferentially to programmed cell death (apoptosis). Since the rate of protein biosynthesis is particularly high both in virus-infected and in rapidly dividing tumor cells, such cells in particular will react strongly to the action of inhibitors of the UPS and of protein folding, whereas normal and healthy cells will remain very largely unaffected by these inhibitors. It is on this principle that the fundamental mechanism of action of the new therapeutic method proposed according to the invention is based.

In a particular embodiment of the invention, the effect of these inhibitors is used for treatment of plasmacytoma cells of patients with multiple myeloma. These B-cell tumors are characterized by an extremely high rate of synthesis of immunoglobulins. It is known that these plasmacytoma cells are particularly sensitive to treatment with proteasome inhibitors. Thus proteasome inhibitors, especially in the form of boric acid peptides (trade name Velcade) have been used successfully for the treatment of multiple myeloma. Nevertheless, it must be kept in mind that there is a very narrow therapeutic window for treatment with proteasome inhibitors, since the boundary between the therapeutic dose and the tolerable toxic dose is very narrow. By virtue of the treatment with inhibitors of protein folding, such plasmacytoma cells are sensitized for action on proteasome inhibitors. The combination of proteasome inhibitors and inhibitors of protein folding causes the effect of both active ingredients to be potentiated synergistically. At the same time, the two medications can be used in sub-toxic doses with higher efficacy, thus in total substantially increasing the prospects for success of the therapy.

The inventive solution offers the following advantages compared with the prior art:
avoidance of resistances
curing of certain diseases
higher responder rate
treatment of several tumor forms (mild, moderate, severe cases)

A further preferred embodiment of the invention relates to the anti-viral action when the two active ingredients are combined. It is known that proteasome inhibitors interfere with the replication of human immune-deficiency viruses (HIV) and other viruses, inducing accumulation of improperly folded Gag proteins, thus interfering with the orderly processes of assembly and release of progeny viruses. This therapeutic action of proteasome inhibitors is greatly potentiated when the virus-infected cell is simultaneously treated with inhibitors of protein folding. Thereby the number of improperly folded structural proteins of the virus is increased, thus intensively interfering with the assembly of viral proteins and thereby the formation of progeny viruses in a trans-negative mechanism, or in other words a prion-like mode of action. This embodiment of the invention is generally valid for all viral infections in which orderly assembly of resynthesized viral structural proteins occurs.

The invention will be explained in more detail on the basis of exemplary embodiments, without being limited to these examples.

### EXAMPLES

### Example 1

The Hsp90 inhibitor 17-AAG in a concentration of up to 10 nM does not exhibit any cytotoxicity in CEM cells.

CD4⁺ T lymph cells (CEM cells) were seeded into a 96-well plate in a density of 1×10⁴ cells per 100 μL. Appropriate amounts of 17-AAG were added to the medium beforehand (see Example 4a), to reach final concentrations of 1 μM, 100 nM, 10 nM, 1 nM, 0.1 nM and 0.01 nM of 17-AAG. After 30 hours of incubation at 37° C. and 5% CO₂, 10 μL of AlamarBlue™ (Invitrogen) was added and all preparations were incubated at 37° C. for a further 4 hours. It was possible to determine a criterion for the viability of the CEM cells (reported in MTT CEM) under the influence of 17-AAG by measuring the color change of the medium using fluorescence measurement at 530/590 nm. Triplicate preparations were used in all cases.

### Example 2

Under the influence of 17-AAG, HeLaSS6 cells transfected with pNLenv1 exhibit reduced Gag processing in the virus fraction and intensified Hsp70 expression in the cell fraction.

Time kinetics were studied for biochemical analysis of the influence of 17-AAG on the kinetics of Gag processing and virus release. The experimental details of cultivation, transfection, media exchange and time kinetics are reported in Example 4a/b. For this purpose there were used cultures of HeLaSS6 cells that had been transfected with pNLenv1 (Schubert et al., 1995). Following incubation in 17-AAG-containing medium (100 nM 17-AAG) or inhibitor-free medium, the kinetic studies were begun after distinct washing steps and aliquoting of the preparations. Aliquot cell cultures were taken at each time and separated into cell, virus and cell-culture supernatant fractions by centrifugation. The HIV proteins were separated by SDS PAGE, transferred onto PVDF membranes and then made visible on x-ray films by antibody-mediated chemiluminescence.

### Example 3

17-AAG and also the combination with PS341 inhibits the virus replication of X4-trophic HI viruses in the HLAC model.

Human tonsils were macerated and transferred into 96-well plates. After one day of incubation, the cells were infected with X4-trophic HI viruses, mixed with the corresponding inhibitors and washed on the following day. These and also the subsequent steps are described in detail under Example 4c-d. At each kinetic point, 150 μL of medium was removed and stored at −80° C. until measurement at RT. The medium that was again added contained the inhibitor concentrations necessary for the special preparation.

After 15 days, the proportion of functional HI viruses formed was determined by means of RT assays (see Example 4e) of the stored supernatants.

### Example 4

### Material and Methods

### Example 4a

### Cell Culture

CEM cells were cultivated in RPMI 1640 with 10% (V/V) fetal calf serum, 2 mM L-glutamine, 100 U/mL penicillin and 100 μg/mL streptomycin.

HeLa cells (ATCC CCL2) were cultivated in Dulbeccos' modified Eagle's medium (DMEM) with 10% fetal calf serum, 2 mM L-glutamine, 100 U/mL penicillin and 100 μg/mL streptomycin.

Tonsil cells were cultivated in RPMI 1640 with 15% (V/V) fetal calf serum, 2 mM L-glutamine, 100 U/mL penicillin, 100 μg/mL streptomycin, 2.5 μg/mL Fungizone, 1 mM sodium pyrovate, 1% MEM non-essential amino acid solution and 50 μg/mL gentanamycin (“tonsil medium”).

### Example 4b

### Transfection, Media Exchange and Kinetics

HeLa cells (ATCC CCL2) were transfected using a mixture of pNLΔenv and lipofectamine2000 in OPTI-MEM. A media exchange was undertaken after 8 hours of incubation at 37° C. and 5% CO₂. In one of the two preparations, a final concentration of 100 nM 17-AAG was added to the medium, which was incubated for a further 16 hours. After distinct washing steps in PBS, aliquots were taken at the corresponding times. At the corresponding times, the cells were separated from the supernatant by centrifuging (5 minutes; 5000 rpm) and later were lyzed by means of CHAPS/DOC lysis (3 minutes on ice). The VLPs in the supernatant were pelleted over a 20% sucrose cushion (90 minutes; 14000 rpm) and, in the same way as the lyzates of the cell pellets, were separated by means of 10% SDS PAGE, transferred by wet blot to PVDF membranes and blocked in 10% milk powder (in PBS/0.1% Tween). The HIV-specific and cell-specific proteins were detected via specific antibodies (to Hsp70; Hsp90; p24; PR55; β-actin). By means of reaction with secondary antibodies and their coupled chemiluminescence, it was possible to detect the signals on x-ray films.

### Example 4c

### Transfection and Extraction of Virus Stocks

To produce virus preparations, plasmid DNA of molecular HIV-1 DNA was transfected into HeLa cells using the calcium phosphate precipitation method. For this purpose, confluent cultures of HeLa cells (5×10⁶ cells) were incubated with 25 μg of plasmid DNA in calcium phosphate crystals, produced according to a method of Graham and van der Eb (1973), then subjected to glycerol shock according to Gorman et al. (1982). To obtain concentrated virus preparations, the cell culture supernatants were harvested two days after transfection. Thereafter the cells as well as their constituents were separated by centrifugation (1000 g, 5 minutes, 4° C.) and filtration (0.45 μm pore size). Virus particles were pelleted by ultracentrifugation (Beckman SW55 rotor, 1.5 hours, 35,000 rpm, 10° C.) and then resuspended in 1 mL of DMEM medium. The virus preparations were sterilized by filtration (0.45 μm pore size) and were frozen in portions (−80° C.). Individual virus preparations were standardized by determination of the reverse transcriptase activity, specifically on the basis of an already described test (Willey et al., 1988), using [32P]-TTP incorporation into an oligo(dT)-poly(A) template.

### Example 4d

### HLAC Model (Extraction, Infection, Kinetics)

The tonsil tissue was washed in PBS, then cleaned of blood clots and cut into pieces measuring 1 to 2 mm² with the scalpel. Individual cells were obtained by mechanical pressing through a filter gauze. Following centrifugation of the isolated cells (5 minutes, 1200 rpm), the cells were counted, seeded into 96-well plates and incubated overnight at 37° C. and 5% CO₂. Infection of the cells was achieved by addition of 10 ng of X4-trophic HIV stocks and simultaneous application of the corresponding inhibitor concentrations. On the following day, 50 μL of supernatant was withdrawn (“1 dpi”) and stored at −80° C. Thereupon the cells were centrifuged (5 minutes, 1200 rpm) and a further 50 μL of supernatant was withdrawn. Following resuspension of the cells in 100 μL of tonsil medium, this washing step was repeated two times. Tonsil medium with the corresponding inhibitor concentrations was added and then the cells were re-incubated at 37° C. and 5% CO₂. On days 3, 6, 9 and 12, 150 μL of medium was withdrawn and stored at −80° C., and 150 μL of medium with the corresponding inhibitor concentrations was added. On day 15, only 150 μL of supernatant was removed and stored at −80° C., after which the cells were discarded.

### Example 4e

### RT Assay

The tonsil supernatants stored at −80° C. were assayed by determination of the reverse transcriptase activity, specifically on the basis of an already described test (Willey et al., 1988), using [32P]-TTP incorporation into an oligo(dT)-poly(A) template.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

One embodiment is a method of treating a viral disease in a subject in need of such treatment, the method comprising administering an effective amount of at least one inhibitor of a ubiquitin-proteasome system and at least one inhibitor of a protein-folding enzyme to treat said viral disease.

Another embodiment is that the at least inhibitor of protein-folding enzymes is at least one inhibitor of cellular chaperones.

Another embodiment is that administering comprising oral, intravenous, intramuscular or subcutaneous administration.

Another embodiment is that the at least one inhibitor of a protein folding enzyme is geldanamycin, radicicol, deoxyspergualin, 4-phenyl butyrate, herbimycin A, epolactaene, Scythe and Reaper, artemisinin, CCT0180159, SNX-2112, radanamycin, novobiocin, or quercetin.

Another embodiment is that the viral disease is caused by HIV-1, HIV-2, hepatitis C virus, hepatitis B virus, Severe Acute Respiratory Syndrome corona virus, smallpox, Ebola virus, influenza virus.

Another embodiment is that cyclosporin A and/or tacrolimus are also administered.

Another embodiment is that the at least one inhibitor of the ubiquitin-proteasome system is

a proteasome inhibitor of a complete 26S proteosome complex and free 20S catalytically active proteasome structure that is not assembled with regulatory subunits,
a ubiquitin ligase inhibitor,
a ubiquitin hydrolase inhibitor,
a ubiquitin-activating enzyme inhibitor,
a mono-ubiquitinylation inhibitor, or
a poly-ubiquitinylation inhibitor.

Another embodiment is that the at least one inhibitor of the ubiquitin-proteasome system is a peptide compound that contains a C-terminal epoxyketone structure, a β-lactone compound, aclacinomycin A, lactacystine, or clasto-lactacysteine β-lactone.

Another embodiment is that the at least one inhibitor of the ubiquitin-proteasome system is N-carbobenzoxy-L-leucinyl-L-leucinyl-L-leucinal, MG232; N-carbobenzoxy-Leu-Leu-Nva-H, N-acetyl-L-leucinyl-L-leucinyl-L-norleucinal, or N-carbobenzoxy-Ile-Glu(OBut)-Ala-Leu-H.

Another embodiment is that the at least one inhibitor of the ubiquitin-proteasome system is a peptide that contains a C-terminal α,β-epoxyketone structures, or a vinylsulfone selected from as carbobenzoxy-L-leucinyl-L-leucinyl-L-leucine vinylsulfone and 4-hydroxy-5-iodo-3-nitrophenylactetyl-L-leucinyl-L-leucinyl-L-leucine vinylsulfone.

Another embodiment is that the at least one inhibitor of the ubiquitin-proteasome system is pyrazyl-CONH(CHPhe)CONH(CHisobutyl)B(OH)₂), dipeptidyl-boric acid ester, or benzyloxycarbonyl(Cbz)-Leu-Leu-boroLeu pinacol ester.

Another embodiment is that the at least one inhibitor of the ubiquitin-proteasome system is epoxomycin and/or eponemycin.

Another embodiment is that the at least one inhibitor of the ubiquitin-proteasome system is one or more of

PS-519 and 1R-[1S,4R,5S]]-1-(1-hydroxy-2-methylpropyl)-4-propyl-6-oxa-2-azabicyclo[3.2.0]heptane-3,7-dione,
PS-314 and N-pyrazinecarbonyl-L-phenylalanin-L-leucine boric acid,
PS-273 (morpholin-CONH—(CH-naphthyl)-CONH—(CH-isobutyl)-B(OH)₂) and its enantiomer PS-293,
PS-296 (8-quinolyl-sulfonyl-CONH—(CH-napthyl)-CONH(—CH-isobutyl)-B(OH)₂),

### PS-303 (NH₂(CH-naphthyl)-CONH—(CH-isobutyl)-B(OH)₂),

PS-321 as (morpholin-CONH—(CH-napthyl)-CONH—(CH-phenylalanin)-B(OH)₂),

### PS-334 (CH₃—NH—(CH-naphthyl-CONH—(CH-isobutyl)-B(OH)₂),

PS-325 (2-quinol-CONH—(CH-homo-phenylalanin)-CONH—(CH-isobutyl)-B(OH)₂),
PS-352 (phenyalanin-CH₂—CH₂—CONH—(CH-phenylalanin)-CONH—(CH-isobutyl)-1-B(OH)₂), and
PS-383 (pyridyl-CONH—(CHρF-phenylalanin)-CONH—(CH-isobutyl)-B(OH)₂).

Another embodiment is that the viral disease is caused by a lymphoma virus, herpes simplex virus, cytomegalovirus, varicella zoster virus, measles virus, Lassa fever virus, paramyxovirus, encephalitis virus, hepatitis A virus, Hepatitis D virus, hepatitis E virus, hepatitis G virus, German measles virus, Coxsackie B virus, or polio virus.

Another embodiment is that the viral disease is measles, Lassa fever, AIDS Mumps, meningitis, orchitis, enteritis; flu, encephalitis, hepatitis, German measles, Poliomyelitis, encephalomyelitis, pancreatitis, pneumonia, myocarditis, or tropical viral disease.

Another embodiment is that the subject is human.

Another embodiment is that the subject has an acute viral infection.

Another embodiment is that the subject has a chronic viral infection.

1. A method of treating a viral disease in a subject in need of such treatment, the method comprising administering an effective amount of at least one inhibitor of a ubiquitin-proteasome system and at least one inhibitor of a protein-folding enzyme to treat said viral disease.

2. The method of claim 1, wherein the at least inhibitor of protein-folding enzymes is at least one chemical anti-chaperone.

3. The method of claim 2, wherein the at least one chemical anti-chaperone is glycerol, trimethylamine, betaine, trehalose or deuterated water (D₂O).

4. The method of claim 1, wherein the at least inhibitor of protein-folding enzymes is at least one inhibitor of cellular chaperones.

5. The method of claim 1, wherein administering comprising oral, intravenous, intramuscular or subcutaneous administration.

6. The method of claim 1, wherein the at least one inhibitor of a protein folding enzyme is geldanamycin, radicicol, deoxyspergualin, 4-phenyl butyrate, herbimycin A, epolactaene, Scythe and Reaper, artemisinin, CCT0180159, SNX-2112, radanamycin, novobiocin, or quercetin.

7. The method of claim 1, wherein the viral disease is caused by HIV-1, HIV-2, hepatitis C virus, hepatitis B virus, Severe Acute Respiratory Syndrome corona virus, smallpox, Ebola virus, influenza virus, or viral hemorrhagic fever.

8. The method of claim 1, wherein the viral disease is caused by Influenza A.

9. The method of claim 1, further comprising administering cyclosporin A and/or tacrolimus.

10. The method of claim 1, wherein the at least one inhibitor of the ubiquitin-proteasome system is
a) a proteasome inhibitor of a complete 26S proteosome complex and free 20S catalytically active proteasome structure that is not assembled with regulatory subunits,
b) a ubiquitin ligase inhibitor,
c) a ubiquitin hydrolase inhibitor,
d) a ubiquitin-activating enzyme inhibitor,
e) a mono-ubiquitinylation inhibitor, or
f) a poly-ubiquitinylation inhibitor.

11. The method of claim 1, wherein the at least one inhibitor of the ubiquitin-proteasome system is a peptide compound that contains a C-terminal epoxyketone structure, a β-lactone compound, aclacinomycin A, lactacystine, or clasto-lactacysteine β-lactone.

12. The method of claim 1, wherein the at least one inhibitor of the ubiquitin-proteasome system is N-carbobenzoxy-L-leucinyl-L-leucinyl-L-leucinal, MG232; N-carbobenzoxy-Leu-Leu-Nva-H, N-acetyl-L-leucinyl-L-leucinyl-L-norleucinal, or N-carbobenzoxy-Ile-Glu(OBut)-Ala-Leu-H.

13. The method of claim 1, wherein the at least one inhibitor of the ubiquitin-proteasome system is a peptide that contains a C-terminal α,β-epoxyketone structures, or a vinylsulfone selected from as carbobenzoxy-L-leucinyl-L-leucinyl-L-leucine vinylsulfone and 4-hydroxy-5-iodo-3-nitrophenylactetyl-L-leucinyl-L-leucinyl-L-leucine vinylsulfone.

14. The method of claim 1, wherein the at least one inhibitor of the ubiquitin-proteasome system is pyrazyl-CONH(CHPhe)CONH(CHisobutyl)B(OH)₂), dipeptidyl-boric acid ester, or benzyloxycarbonyl(Cbz)-Leu-Leu-boroLeu pinacol ester.

15. The method of claim 1, wherein the at least one inhibitor of the ubiquitin-proteasome system is epoxomycin and/or eponemycin.

16. The method of claim 1, wherein the at least one inhibitor of the ubiquitin-proteasome system is one or more of
PS-519 and 1R-[1S,4R,5S]]-1-(1-hydroxy-2-methylpropyl)-4-propyl-6-oxa-2-azabicyclo[3.2.0]heptane-3,7-dione,
PS-314 and N-pyrazinecarbonyl-L-phenylalanin-L-leucine boric acid,
PS-273 (morpholin-CONH—(CH-naphthyl)-CONH—(CH-isobutyl)-B(OH)₂) and its enantiomer PS-293,
PS-296 (8-quinolyl-sulfonyl-CONH—(CH-napthyl)-CONH(—CH-isobutyl)-B(OH)₂),
PS-303 (NH₂(CH-naphthyl)-CONH—(CH-isobutyl)-B(OH)₂),
PS-321 as (morpholin-CONH—(CH-napthyl)-CONH—(CH-phenylalanin)-B(OH)₂),
PS-334 (CH₃-NH—(CH-naphthyl-CONH—(CH-isobutyl)-B(OH)₂),
PS-325 (2-quinol-CONH—(CH-homo-phenylalanin)-CONH—(CH-isobutyl)-B(OH)₂),
PS-352 (phenyalanin-CH₂—CH₂—CONH—(CH-phenylalanin)-CONH—(CH-isobutyl)-1-B(OH)₂), and
PS-383 (pyridyl-CONH—(CHρF-phenylalanin)-CONH—(CH-isobutyl)-B(OH)₂).

17. The method of claim 1, wherein the viral disease is caused by a lymphoma virus, herpes simplex virus, cytomegalovirus, varicella zoster virus, measles virus, Lassa fever virus, paramyxovirus, encephalitis virus, hepatitis A virus, Hepatitis D virus, hepatitis E virus, hepatitis G virus, German measles virus, Coxsackie B virus, or polio virus.

18. The method of claim 1, wherein the viral disease is measles, Lassa fever, AIDS Mumps, meningitis, orchitis, enteritis; flu, encephalitis, hepatitis, German measles, Poliomyelitis, encephalomyelitis, pancreatitis, pneumonia, myocarditis, or tropical viral disease.

19. The method of claim 1, wherein the subject is human.

20. The method of claim 1, wherein the subject has an acute viral infection.

21. The method of claim 1, wherein the subject has a chronic viral infection.

22. The method of claim 1, which comprises administering the at least one inhibitor in combination with other agents that are used for treatment of viral infections and/or tumor diseases.

23. The method of claim 1, wherein the at least one inhibitor is in a composition in the form of inhalations, depot forms, plasters, or microelectronic systems.

24. The method of claim 1, wherein the viral disease is caused by a virus infection:
head, neck CA,
epithelial CA,
ovarian CA,
colon CA,
leukemia (AML, ALL, CML, CLL),
acute myeloic, chronic,
acute lymphatic, chronic,
lymphoma (non-Hodgkins),
cervical Ca,
cervical Ca,
metastases (such as bone marrow),
lymphoma viruses,
herpes simplex,
cytomegaly,
varicella zoster,
Varicella Zoster,
measles,
Lassa fever,
AIDS,
mumps (-meningitis, -orchitis),
enteritis; flu (all forms),
encephalitis,
hepatitis (A, B, C, D, E, G),
German measles,
Coxsackie B,
polio (-myelitis),
encephalomyelitis,
pancreatitis,
pneumonia,
myocarditis,
tropical diseases (viral), or
all double-strand and single-strand DNA and RNA viruses that are pathogenic for humans.
